(19) United States
(12) Patent Application Publication
(10) Pub. No.: US 2018/0276345 A1
(43) Pub. Date: Sep. 27, 2018

(54) SYSTEM AND METHOD TO MONITOR MENTAL HEALTH IMPLICATIONS OF UNHEALTHY BEHAVIOR AND OPTIMIZE MENTAL AND PHYSICAL HEALTH VIA A MOBILE DEVICE

(71) Applicant: INTERNATIONAL BUSINESS MACHINES CORPORATION, ARMONK, NY (US)

(72) Inventors: STEFAN VON CAVALLAR, SANDRINGHAM (AU); TIMOTHY M. LYNAR, KEW (AU); JORGE ANDRES MOROS ORTIZ, CARLTON (AU); JOHN M. WAGNER, CARLTON (AU)

(21) Appl. No.: 15/468,811

(22) Filed: Mar. 24, 2017

(51) Int. Cl. G06F 19/00; G06N 99/00; A61B 5/16

(52) U.S. Cl. CPC G06F 19/3418; G06F 19/322; G06F 19/324; G06Q 30/0201; A61B 5/165; G06F 19/3437; G06N 99/005

(57) ABSTRACT

A method including: receiving physiological and external data of a user; predicting that the user is gravitating towards an undesirable mental state based on the physiological and external data; and providing the user with an ameliorative action in response to the prediction that the user is gravitating towards the undesirable mental state.

### BACKGROUND

The present invention relates to a system and method to monitor mental health of users, and more specifically, to execute context sensitive and cognitively sensitive mitigation strategies and one or more ameliorative actions to optimize mental or physical health outcomes via a mobile device.

Looking at alternatives to and to prevent unhealthy behavior by informing and influencing people who are likely to conduct such behaviors can be beneficial for their mental health, and for the mental health of those around them. In today's world, the large volume of data available can give insights to preventing and supporting an individual's mental health. Currently, the annual costs in the United States associated with mental health are estimated at $2.5 trillion USD.

With the plethora of hand-held devices, wearables, and environment and social data monitoring capabilities, it is possible to monitor, study and predict a user's mental health status. Consequently, ameliorative actions and mitigation strategies can be deployed to a user who may start to gravitate towards a given emotional state.

### SUMMARY

According to an exemplary embodiment of the present invention, there is provided a method including: receiving physiological and external data of a user; predicting that the user is gravitating towards an undesirable mental state based on the physiological and external data; and providing the user with an ameliorative action in response to the prediction that the user is gravitating towards the undesirable mental state.

According to an exemplary embodiment of the present invention, there is provided a method including: collecting medical records, social graph interactions, media analysis and spending habits of a user; collecting, from a mobile device or a personal sensor, physiological, location and financial data of the user; creating a model of the user and the user's interactions over time; detecting identifiers in the model through supervised learning; learning through a supervised approach which identifiers are positive or negative; and sending an alert when an initiation of an identified negative pattern is detected, and initiating a predefined ameliorative action.

According to an exemplary embodiment of the present invention, there is provided a method including: receiving physiological data of a user and data about an area where the user is located; determining that the user is gravitating towards a negative emotional state by inferring negative identifiers from the physiological data of the user and the data about an area where the user is located; and alerting the user that they are gravitating towards the negative emotional state.

### BRIEF DESCRIPTION OF THE DRAWINGS

FIG. 1 illustrates a method according to an exemplary embodiment of the present invention;

FIG. 2 illustrates a system according to an exemplary embodiment of the present invention;

FIG. 3 illustrates a method according to an exemplary embodiment of the present invention; and

FIG. 4 illustrates an apparatus for implementing an exemplary embodiment of the present invention.

### DETAILED DESCRIPTION

In accordance with an exemplary embodiment of the present invention, there is provided a system and method to monitor mental health implications of unhealthy behavior and optimize mental/physical health outcomes via a mobile device.

By utilizing the plethora of hand-held devices, wearables, and environment and social data monitoring capabilities, the present invention provides a system and method with the ability to monitor, model and predict a user's mental health status. The present invention may be hereinafter referred to interchangeably as “the system” or “the method.” The system deploys ameliorative actions and mitigation strategies on a user who may start to gravitate towards a given state.

Ameliorative actions that are executed on a user can include, but are not limited to, suggestions ranging from simple suggestions such as music to listen to, places to visit and events to attend, to more complex actions such as scheduling visits with friends, recommending foods to eat, suggesting alternative routes on maps, or future yet unknown hand-held device interactions that can provide various options to the user. This way, for example, the system will assist the user in selecting, according to the system's understanding of the user's state, to rebalance their mood if needed.

Mitigation strategies are selected in advance with consultation of an expert system and agreement with the user. Mitigated strategies may be stepped. These strategies can include, but are not limited to, limiting communication in terms of quantity of communications, tempo of communications, destination for communications, and or content of communications; limiting financial transactions by way of destination of payment, value of payments, types of payments, frequency of payments, or a combination of any or all of the aforementioned; sending escalating alerts to predefined contacts based on the behavior of the monitored individual; issuing alarms, alerts, or distractions based on geographical location.

By modeling the user in 360-degree view, the collected data can provide insight to support improving their mood when needed and be presented in a non-intrusive way. The present invention can suggest to the user which alternatives can be more beneficial for supporting their mental health. The present invention can be overlaid with existing systems as a supplemental function or as an enhancement, for example. In addition, the present invention presents information in a multi-device accessible portal to provide awareness, and support this with following actions for change.

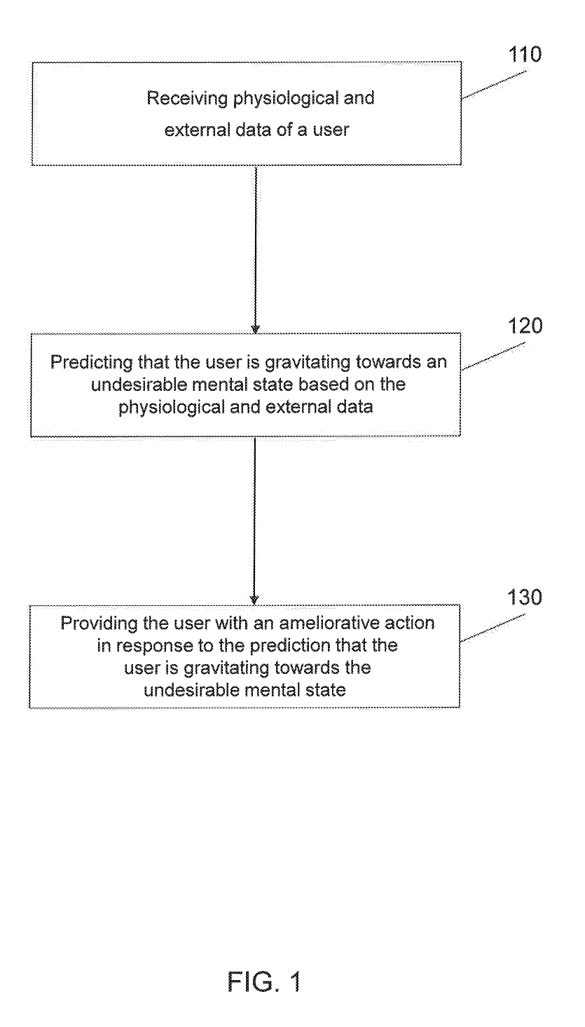

A method according to an exemplary embodiment of the present invention will now be described with reference to FIG. 1.

In the method, a computer or a computing system operating in accordance with an exemplary embodiment of the present invention will receive physiological and external data of a user (110). As an example, this data can be provided wirelessly from an electronic device possessed by the user to the computer. For example, this data may be transmitted from the user's smartphone to the computer via cellular network. In response to the receipt of this data, the computer can predict whether the user is gravitating towards an undesirable emotional state or not (120). The details of this step will be described later. If it is determined that the user is gravitating towards the undesirable emotional state, the computer can provide the user with an ameliorative action (130). Example ameliorative actions will be described later.

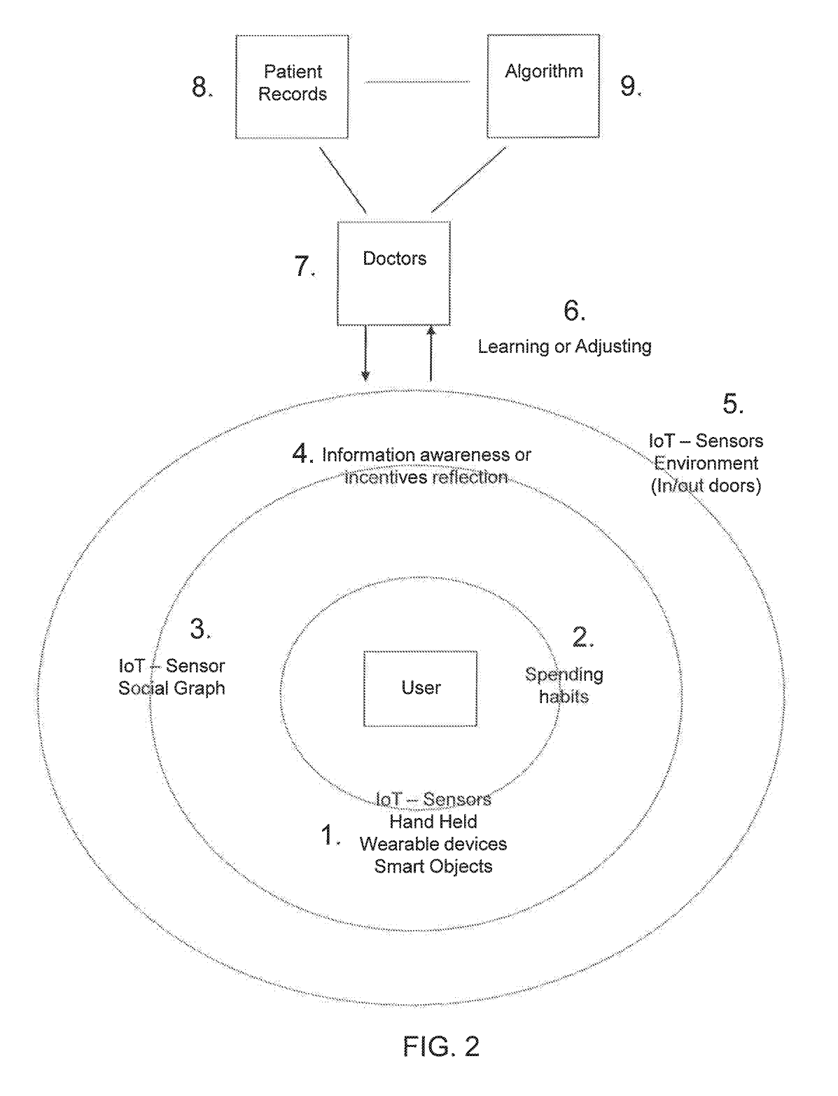

FIG. 2 illustrates a system according to an exemplary embodiment of the present invention. In particular, FIG. 2 illustrates certain components of the present invention. For example, as shown in FIG. 2, there is provided Internet of Things (IoT) sensors, Hand Held and Wearables and Smart Objects (1, 3 and 5).

The elements indicated by reference numerals (1, 3 and 5) may refer to sensors that are connected to a broader network, whereby each sensor may monitor a specific aspect. Networks are indicated by dashed circles in FIG. 2. For example, smaller circles may indicate personal networks, while a larger circle may indicate a network of inter-connected devices. The aggregation of all sensors may form a more overall/holistic view. For example, one sensor may sense a user's temperature, while another measures movement in space, and another focuses on location. The aggregation of the sensors can show the temporal change of the user's temperature. Additionally, the movement and the location of the activity can also be shown to present greater meaning.

IoT sensors are also employed to sense physiological signs that are known for when a user is starting to become stimulated from emotional state changes. This information can be fed back into the system to first understand the benchmark of an individual in terms of emotional thresholds and changes. This information also serves as an alert for the system to indicate that a change may occur, and, when in an emotional state, to gauge its severity in relation to previous situations.

Similarly, wearable devices (e.g., activity trackers), and smart objects (e.g., devices interacting via radio frequency identification (RFID), Bluetooth low energy (BLE) or near field communication (NFC)) may sense extra data points, such as typing speed, sweat on hands, clumsiness via video, etc. These data add to the broader understanding of a user's context.

FIG. 2 shows spending habits of the user (2). This may be the analysis of monetary spending (e.g., withdrawals from ATMs), in relation to events, people the user spends time with, and emotional states of the user. The present invention can predict how a user's spending may affect/improve their emotional state. For example, if one of the user's patterns is regular spending for alcohol when they feel depressed or are in a state conducive to risky behavior, the system can run prediction models and suggest interventions to assist the user in refraining from that behavior.

FIG. 2 shows an IoT sensor social graph (3). The social graph represents a user's network—their peers and people they spend time with, their likes, relationships and comments (via Facebook, for example). The graph data can be used to find alternative suggestions to the user's routine, such as events, walks, music, etc. It is known that peer recommended activities are a form of “trust transfer”, and are more effective than suggesting a singular activity (e.g., suggesting a nice café). Instead, the present invention may suggest, “you visited this place a while back, and you had X food with Y person, the place is located nearby, they have a new menu, you may like it”. The graph offers via Natural Language Processing (NLP) understanding as to why the user may have a changed state. Take the following scenario: A user receives a break-up message from their partner via Facebook, leading him to heavy drinking, partying and driving dangerously. The present invention can predict this event and suggest an activity with a close friend, for example.

FIG. 2 shows information awareness or incentives reflection (4). This part of the diagram refers to the system triggering interventions. For example, “information awareness” refers to when the system can predict that the user is gravitating to an emotional state that may be conducive of risky behavior. The system then triggers an intervention in relation to what the user is doing, such as suggesting an event, music, a friend near-by, while the user is walking or driving in a certain direction, or it may be suggested for later in the day according to their plans. The “reflection” is mutual in that the system knows the user's emotional state is heading negative, and that the user did not take the suggested options. This serves as an opportunity for the system to gauge the user's response and situation. The other part of the “reflection” is by the user, where the user has chosen to continue in the course of their emotional state. Even if the system only gives the user the ability to reflect upon their state, this can help develop emotional awareness for the user over time.

FIG. 2 shows learning or adjusting (6). Here, a supervised learning technique is employed on the data provided from (1-5). One of the following supervised learning techniques may be used: neural networks, decision tree learning, case-based reasoning, or Naive Bayes classifier.

FIG. 2 shows doctors and patient records (7 and 8). Here, the system ingests and processes patient records, notes and other metadata pertaining to the relevant user to contribute to improved machine learning. The curation of this data may be used to determine a greater “weighting” to its contribution to any predictive analytics carried out by the system.

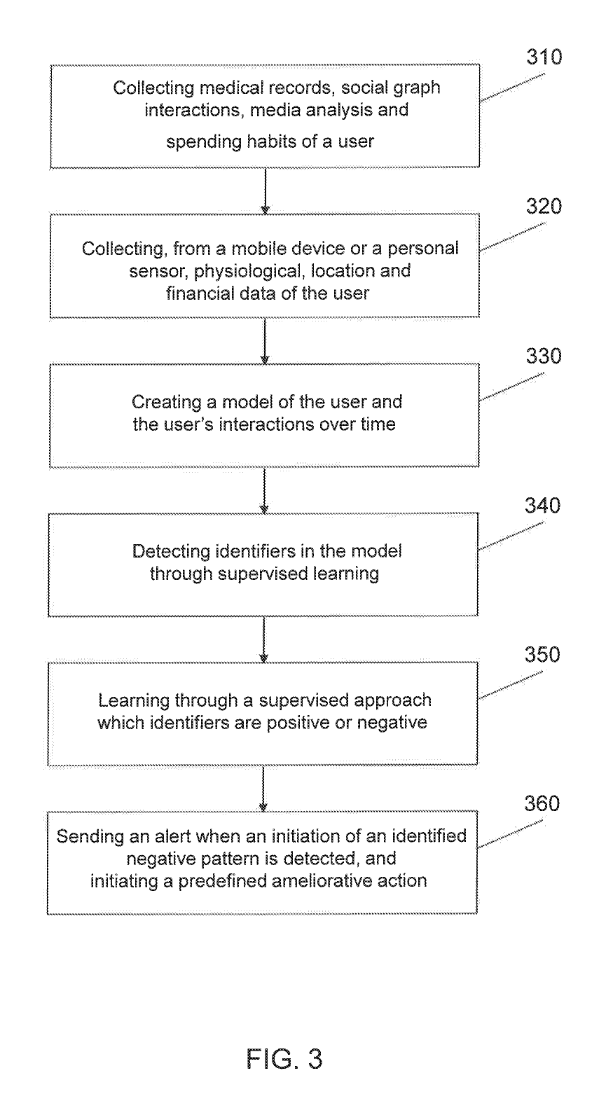

FIG. 2 shows an algorithm that may be performed by a computer or a computer system to carry out processes according to an exemplary embodiment of the present invention (9). The algorithm is shown in greater detail in FIG. 3.

For example, as shown in FIG. 3, there is provided a step of collecting medical records, social graph interactions, media analysis and spending habits of a user (310). FIG. 3 also shows a step of collecting, from a mobile device or a personal sensor, physiological, location and financial data of the user (320). Information collected may include physiological, location, and financial data using the user's devices. Services such as those offered by financial institutions may be accessed to track the user's physical location and financial spending, e.g., record transactions.

Location information may be provided by location services such as Global Positioning System (GPS), Global Navigation Satellite System (GLONAS) or other available location based systems. Financial data may be obtained via the user's digital wallet, smartphone, payment device, financial institution or payment API service. Financial data may also be obtained from another service offered by the user's financial institution or payment device.

As further shown in FIG. 3, a model of the user and the user's interactions over time is created (330). Using the model, identifiers can be detected through supervised learning (340). The identifiers may be, for example, patterns, events, or other properties. Pattern detection and feature detection methods may include those employing a time series analysis. An example of such a method is described in Choi et al., “Applying Machine Learning Methods for Time Series Forecasting,” Proceedings of the ISATED International Conference Artificial Intelligence and Applications (AIA 2009), Feb. 16-18, 2009, Innsbruck, Austria, the disclosure of which is incorporated by reference herein in its entirety. Another method of pattern mining in time series data is described in the Masters Thesis of Caroline Kleist, entitled “Time Series Data Mining Methods: A Review,” submitted to Prof. Dr. Wolfgang Karl Hardie, at the Humboldt University of Berlin School of Business and Economics, Berlin, Mar. 25, 2015, the disclosure of which is incorporated by reference herein in its entirety.

It is to be understood that as the user decides to take on the offered alternatives from the system towards improving their state, these choices, can be used to improve the system. For example, the system may consider the user's tone of voice when accepting an offered alternative, the time when the user accepts the offered alternative, or the place where the user is when they accept the offered alternative. From these factors, the system can learn from the user to fine-tune its delivery of alternatives. In other words, the system model is trained based on the user's selections or interactions, and thus, it can always evolve.

The detected identifiers can then be learned as positive or negative through a supervised approach (350). Here, for example, a supervised learning approach involves a user indicating which detected features or events specified in the time series analysis are positive or negative.

In the event a negative feature or event is detected, an alert can be sent to the user, and a predefined ameliorative action can be initiated (360). An alert may be a text notification, voice notification or activation of an actuator that produces a sensation that draws attention of one or more persons to a predefined negative situation. An ameliorative action may be an automated workflow which is pre-set, and which may include one or many actions that may be executed from a smartphone—such as text message, phone call, preset message or pre-recorded video, or one or more of: 1) degradation or temporary degradation of utility and/or function of one or more electronic devices, degradation of utility and/or function of one or more online services; 2) degradation of utility and/or function of one or more online financial services; and 3) degradation of utility and/or function of one or more social services (e.g., Facebook, Twitter, etc.).

As can be seen, the present invention predicts, rather than acts, as a result of a degenerated state. For example, the present invention uses collected data, applies ongoing automatic monitoring from sensors and predicts before the user falls into a negative mental state. The user is aware of the monitoring. Inline with preventing, the user is presented with options to boost their mood and prevent gravitating towards an undesirable state. The present invention's data sources go beyond mobile, tablet, desktop, and digital assistants from which to collect data. Such other sources include IoT devices, social graph data, spending habits, and ambient sensing.

The present invention provides options to action in recovery. In addition, the present invention presents information and actions inline with gamification and incentivizing principles. The present invention uses data to constantly improve.

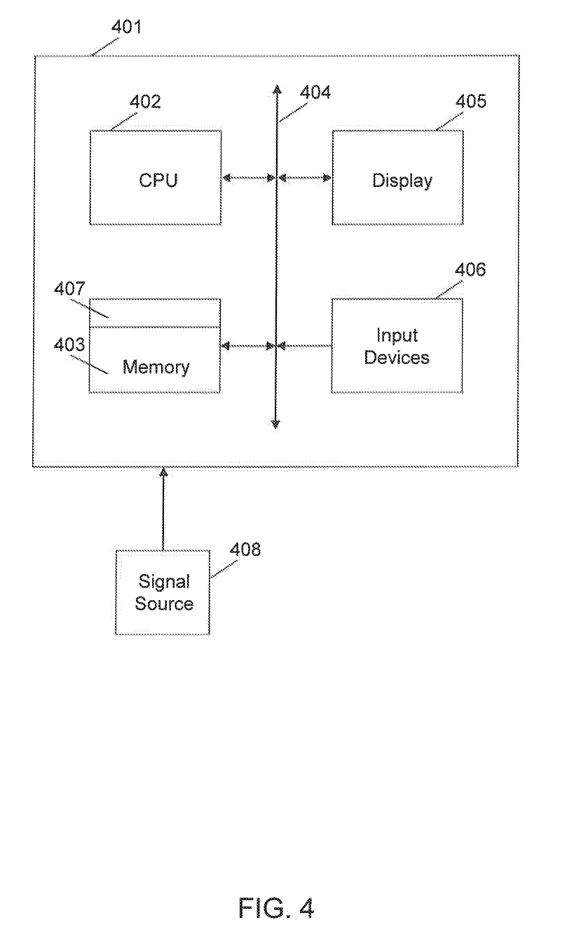

Referring now to FIG. 4, according to an exemplary embodiment of the present invention, a computer system 401 can comprise, inter alia, a CPU 402, a memory 403 and an input/output (I/O) interface 404. The computer system 401 is generally coupled through the I/O interface 404 to a display 405 and various input devices 406 such as a mouse and keyboard. The support circuits can include circuits such as cache, power supplies, clock circuits, and a communications bus. The memory 403 can include RAM, ROM, disk drive, tape drive, etc., or a combination thereof.

Exemplary embodiments of present invention may be implemented as a routine 407 stored in memory 403 (e.g., a non-transitory computer-readable storage medium) and executed by the CPU 402 to process the signal from the signal source 408. As such, the computer system 401 is a general-purpose computer system that becomes a specific purpose computer system when executing the routine 407 of the present invention.

The computer platform 401 also includes an operating system and micro-instruction code. The various processes and functions described herein may either be part of the micro-instruction code or part of the application program (or a combination thereof) which is executed via the operating system. In addition, various other peripheral devices may be connected to the computer platform such as an additional data storage device and a printing device.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++ or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, comprising:
receiving physiological and external data of a user;
predicting that the user is gravitating towards an undesirable mental state based on the physiological and external data; and
providing the user with an ameliorative action in response to the prediction that the user is gravitating towards the undesirable mental state.

2. The method of claim 1, wherein the physiological data is provided from internet-of-things (IOT) sensors.

3. The method of claim 1, wherein the predicting that the user is gravitating towards the undesirable mental state based on the physiological and external data comprises:
comparing the physiological and external data of the user with a baseline obtained from a learned model of the user; and
identifying a potential negative change in the user's mental state when the physiological and external data exceed the baseline by a predetermined threshold.

4. The method of claim 3, wherein the model is learned with a supervised learning technique.

5. The method of claim 1, wherein the ameliorative action includes a suggested action for the user to take.

6. The method of claim 1, wherein the ameliorative action includes implementation of a pre-defined mitigation strategy.

7. The method of claim 6, wherein the pre-defined mitigation strategy includes limiting operations that can be performed on the user's computing device.

8. The method of claim 7, wherein the computing device includes a smartphone.

9. The method of claim 1, further comprising monitoring whether the user has taken the ameliorative action.

10. A method, comprising:
collecting medical records, social graph interactions, media analysis and spending habits of a user;
collecting, from a mobile device or a personal sensor, physiological, location and financial data of the user;
creating a model of the user and the user's interactions over time;
detecting identifiers in the model through supervised learning;
learning through a supervised approach which identifiers are positive or negative; and
sending an alert when an initiation of an identified negative pattern is detected, and initiating a predefined ameliorative action.

11. The method of claim 10, wherein the supervised approach involves the user indicating which identifiers are positive or negative.

12. The method of claim 10, wherein the ameliorative action is a pre-set automated workflow.

13. The method of claim 12, wherein the ameliorative action includes one or more actions executable on the mobile device.

14. A method, comprising:
receiving physiological data of a user and data about an area where the user is located;
determining that the user is gravitating towards a negative emotional state by inferring a negative identifier from the physiological data of the user and the data about an area where the user is located; and
alerting the user that they are gravitating towards the negative emotional state.

15. The method of claim 14, further comprising providing the user with an instruction to perform an ameliorative action to offset the negative emotional state.

16. The method of claim 14, wherein the alert is provided to an electronic device in the user's possession.

17. The method of claim 16, wherein the electronic device includes a smartphone or a wearable device.

18. The method of claim 14, wherein the negative identifier is determined by searching a data source of predetermined negative identifiers.

19. The method of claim 18, wherein the predetermined negative identifiers are obtained through a supervised learning on a model of the user and the user's interactions over time.

20. The method of claim 14, wherein the physiological data of the user and the data about an area where the user is located is wirelessly provided to a monitoring system from an electronic device in the user's possession.
